(19) United States
(12) Patent Application Publication
(10) Pub. No.: US 2009/0221892 A1
(43) Pub. Date: Sep. 3, 2009

(54) Dual Transdermal Analyte Sensor Assembly and Methods of Using the Same

(76) Inventors: Allen J. Brenneman, Goshen, IN (US); Mihailo V. Rebec, Bristol, IN (US)

Correspondence Address: NIXON PEABODY LLP, 300 S. Riverside Plaza, 16th Floor, CHICAGO, IL, 60606, US

(21) Appl. No.: 12/086,563

(22) PCT Filed: Dec. 14, 2006
(86) PCT No.: PCT/US2006/047558

§ 371 Date: Jan. 28, 2009

Related U.S. Application Data
(60) Provisional application No. 60/751,260, filed on Dec. 16, 2005.

(51) Int. Cl. A61B 5/1477

(52) U.S. Cl. 600/365; 600/309

(57) ABSTRACT

A transdermal test sensor assembly adapted to assist in determining at least one analyte concentration of a fluid sample is provided. The test sensor assembly comprises a sensor support, a first test sensor, a second test sensor, a first hydrogel composition, and a second hydrogel composition. The first test sensor couples to the sensor support. The second test sensor couples to the sensor support. The first hydrogel composition is positioned on the first test sensor. The second hydrogel composition is positioned on the second test sensor.

### FIELD OF THE INVENTION

The present invention relates generally to a transdermal test sensor assembly. More particularly, the invention relates to a dual transdermal test sensor assembly adapted to assist in determining a concentration of at least one analyte, where the test sensor assembly has at least two transdermal sensors.

### BACKGROUND OF THE INVENTION

The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check the glucose level in their body fluids to regulate the glucose intake in their diets. The results of such tests may be used to determine what, if any, insulin or other medication needs to be administered. In one type of testing system, test sensors are used to test a fluid such as a sample of blood.

According to some existing techniques, a lancet may be used to pierce a user's skin to draw fluid (e.g., blood) from the user. This fluid is then used with an instrument or meter to determine an analyte (e.g., glucose) concentration. Piercing a user's skin each time an analyte concentration reading is desired is an inconvenient and invasive procedure. Moreover, the procedure is undesirable because of the resulting pain and discomfort often experienced by a user.

One non-invasive method for obtaining a sample for determining an analyte concentration involves using a transdermal sample of one or more analytes found in, for example, interstitial fluid (ISF). In this method, a transdermal test sensor is placed on a user's skin. The transdermal sensor typically includes a hydrogel to facilitate the extraction of the analyte from the user's skin to an analyte-testing instrument or meter. The hydrogel must be sufficiently mechanically and thermally stable to provide a relatively static, reactive, and aqueous conduct between a dermal sampling site and an analyte-testing instrument.

One prior attempt at using a transdermal sensor for analyte testing involves using an iontophoretic test sensor, such as that disclosed in U.S. Pat. No. 6,393,318 to Conn et al. One problem with iontophoretic test sensors is that a user's skin may become irritated by the electrical current that flows between two electrodes required by iontophoretic test sensors.

One problem with existing transdermal test sensors relates to its reliability Transdermal testing typically occurs over a long period of time. Therefore, if a problem occurs with a transdermal test sensor during that time, a large amount of data may be lost. Additionally, a flux rate of an analyte through the user's skin may vary over time. Thus, as the flux rate of the analyte changes, the analyte level determined in testing may not accurately reflect the actual analyte level. Additionally, existing transdermal sensors only measure a single analyte.

Thus, it would be desirable to have a transdermal test sensor that assists in addressing one or more of the above disadvantages.

### SUMMARY OF THE INVENTION

According to one embodiment of the present invention, a transdermal test sensor assembly adapted to assist in determining at least one analyte concentration of a fluid sample is provided. The test sensor assembly comprises a sensor support, a first test sensor, a second test sensor, a first hydrogel composition, and a second hydrogel composition. The first test sensor couples to the sensor support. The second test sensor couples to the sensor support. The first hydrogel composition is positioned on the first test sensor. The second hydrogel composition is positioned on the second test sensor.

According to another embodiment of the present invention, a transdermal analyte-testing assembly adapted to determine a concentration of at least one analyte of a sample is provided. The analyte-testing assembly comprises a sensor support, a first test sensor, a second test sensor, a first hydrogel composition, a second hydrogel composition, and an analyte-testing instrument. The first test sensor couples to the sensor support. The second test sensor couples to the sensor support. The first hydrogel composition is positioned on the first test sensor. The second hydrogel composition is positioned on the second test sensor. The analyte-testing instrument couples to the sensor support. The analyte-testing instrument is adapted to determine a concentration of at least one analyte of a sample.

According to one process of the present invention, a non-invasive method of determining a concentration of at least one analyte in a body fluid is provided. The method provides a dual transdermal test sensor assembly that includes a sensor support, a first test sensor, a first hydrogel composition, a second test sensor, and a second hydrogel composition. The first test sensor and the second test sensor couple to the sensor support. The transdermal sensor assembly contacts an area of skin such that the first hydrogel composition and the second hydrogel composition position between the skin and the test sensor. The analyte-testing instrument couples to the dual transdermal test sensor assembly. The concentration of at least one analyte is determined using the analyte-testing instrument.

The above summary of the present invention is not intended to represent each embodiment, or every aspect, of the present invention. Additional features and benefits of the present invention are apparent from the detailed description and figures set forth below.

### BRIEF DESCRIPTION OF THE DRAWINGS

FIG. 1a is a perspective view of a test sensor assembly according to one embodiment of the present invention.

FIG. 1b is an exploded, perspective view of the test sensor assembly of FIG. 1a.

FIG. 2 is a perspective view of a test sensor assembly of the present invention being coupled to an analyte-testing instrument.

### DESCRIPTION OF ILLUSTRATED EMBODIMENTS

The present invention is directed to a transdermal test sensor assembly adapted to assist in determining a concentration of at least one analyte. The transdermal test sensor assembly has two sensor assemblies.

Transdermal test sensors contain a hydrogel composition, which may serve as an interface between the sensor and the skin. A hydrogel composition is defined herein as a polymer gel. The hydrogel composition generally comprises at least one monomer and a solvent. The solvent is typically substantially biocompatible with the skin. Non-limiting examples of solvents that may be used in the hydrogel composition include water and a water mixture. The amount of water in the hydrogel is generally between about ten to about ninety-five percent (10%-95%) by weight, but may vary depending on the monomer amount and cross linking, as well as the characteristics of the gel desired.

The transdermal test sensor assists in determining the concentration of the desired analyte by using the hydrogel as an osmotic agent to extract the analyte from a fluid such as ISF. Analytes that may be measured include glucose, lipid profiles (e.g., cholesterol, triglycerides, LDL, and HDL), fructose, lactate, or bilirubin. It is contemplated that other analyte concentrations may be determined. One non-limiting example of the transdermal sensor's use is to determine the glucose concentration in a user's ISF.

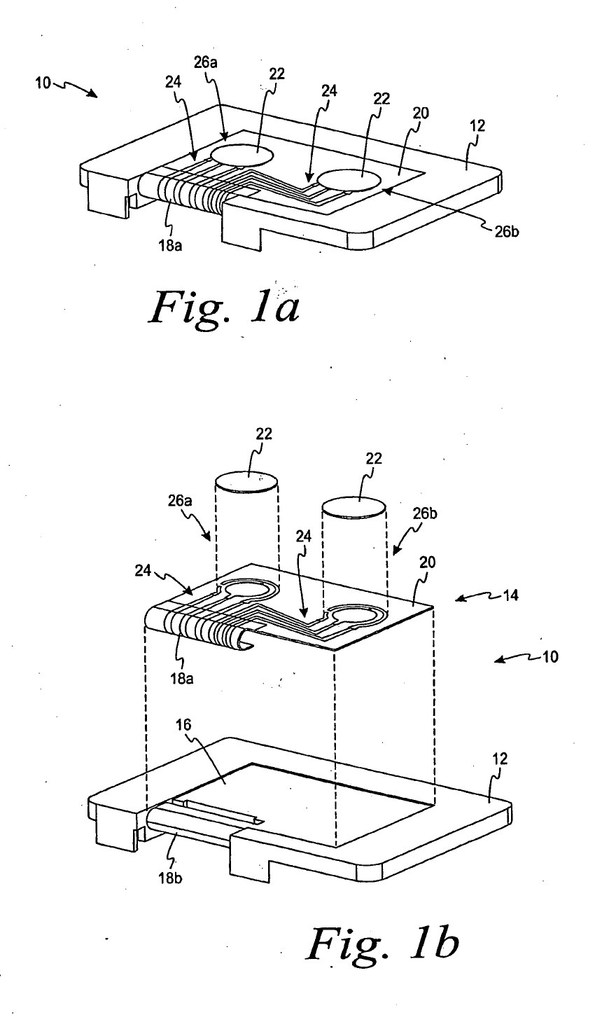

In the embodiment of FIGS. 1a, 1b, a transdermal test sensor assembly 10 is illustrated according to one embodiment of the present invention. Although in this embodiment, the test sensor is an electrochemical sensor, it is contemplated that the present invention may be applied to other sensors (e.g., optical test sensors). An example of an electrochemical sensor includes a standard, three-electrode design utilizing a catalytic, platinum-containing working electrode, a counter electrode and a reference electrode. It is contemplated that other electrochemical sensors may be used including those with fewer electrodes such as a two electrode electrochemical sensor, which includes a counter electrode and a working electrode.

The test sensor assembly 10 includes a sensor support 12 and a test sensor 14. The test sensor 14 is positioned generally parallel and adjacent to the sensor support 12. The sensor support 12 of FIGS. 1a, 1b forms a recessed area 16 having dimensions generally similar to the dimensions of the test sensor 14 to inhibit movement of the test sensor 14 relative to the sensor support 12. It is contemplated that the test sensor assembly of the present invention may include a mechanism to further inhibit movement of the test sensor 14 relative to the sensor support 12. It is desirable for the recessed area 16 to have dimensions substantially similar to the dimensions of the test sensor 14 to inhibit movement of the test sensor 14 relative to the sensor support 12. For example, the test sensor 14 of FIGS. 1a,b includes a flexible element 18a that is adapted to attach to a corresponding curved element 18b of the sensor support 12. It is contemplated that other mechanisms suitable for inhibiting movement of the test sensor 14 with respect to the sensor support 12 may also be used such as positioning an adhesive between the sensor 14 and the sensor support 12. Alternatively, the sensor support 12 may include small plastic molded pins extending from the recessed area 16 through corresponding apertures formed in the test sensor 14. The pins may be, for example, heat stakes or sonic welded to keep the sensor 14 in place.

An outwardly-facing surface 20 of the test sensor 14 includes a hydrogel composition 22. Although in the illustrated embodiment, the hydrogel 22 is generally circular in shape, it is contemplated that the hydrogel 22 may be of any shape. The hydrogel 22 generally has a thickness of from about 0.05 mm to about 5 mm and, more specifically, has a thickness of from about 0.1 mm to about 1 mm. The surface area of the test sensor 14 covered by the hydrogel 22 in one embodiment is from about 0.1 cm2 to about 100 cm2. The hydrogel 22 is generally positioned over a plurality of electrodes 24. The plurality of electrodes 24 includes a counter electrode, a reference electrode, and a working electrode. It is contemplated that other electrode structures may be used.

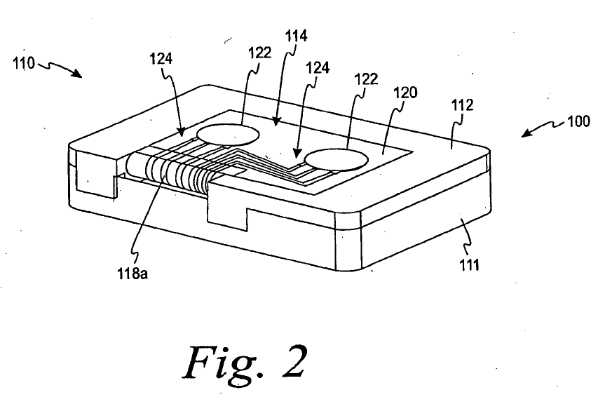

The test sensor 14 is a dual test sensor, wherein each sensor 26a, 26b is independent of the other. It is contemplated that more than two test sensors may be used. The test sensor assembly of the present invention may be coupled to an analyte-testing instrument, or meter, as shown in the embodiment of FIG. 2. Referring to FIG. 2, a meter assembly 100 includes a test sensor assembly 110 coupled to a meter 111. The test sensor assembly 110 of FIG. 2 is substantially similar to the test sensor assembly 10 of FIGS. 1a, 1b described above. In the illustrated embodiment, the meter 111 is coupled to a surface of a sensor support 112 opposite a test dual sensor 114. It is contemplated that the meter 111 may be coupled to other portions of the test sensor assembly 110. It is contemplated that any mechanism suitable for maintaining the test sensor assembly 110 and the meter 111 in a substantially fixed position may be used including, but not limited to, snaps, screws, or other fasteners. The meter 111 is adapted to determine the concentration of the desired analyte extracted from a fluid sample such as an ISF sample.

To test an analyte (e.g., glucose) concentration in an ISF sample, a hydrogel composition 122 on the test sensor 114 is placed against a user's skin, thereby coupling the skin and the test sensor 114. The test sensor assembly 110 further has a plurality of electrodes 124 for each test sensor. The test sensor assembly 110 may be applied at a skin site such as the volar forearm between the wrist and elbow such that the hydrogel 122 is positioned generally between the skin site and the test sensor 114. It is contemplated that the test sensor assembly 110 may be applied at other skin sites such as the abdomen. It is contemplated that the meter 111 and the test sensor assembly 110 may be sued for continual glucose monitoring or for non-continual glucose monitoring.

It may be desirable for the skin to be pre-treated to increase the skin permeability prior to applying the test sensor assembly 110. One example of pre-treating is to use ultrasound energy to disrupt the lipid bilayer of the stratum corneum so as to increase the skin permeability. By increasing the skin permeability, the amount of ISF used in transdermal sampling is increased. This results in improved sampling of the analytes of interest found in the ISF.

One non-limiting source of an ultrasound energy system is Sontra SonoPrep® ultrasonic skin permeation system marketed by Sontra Medical Corporation (Franklin, Mass.). The SonoPrep® system applies relatively low frequency ultrasonic energy to the skin for a limited duration (from about 10 to 20 seconds). The ultrasonic horn contained in the device vibrates at about 55,000 times per second (55 KHz) and applies energy to the skin through the liquid-coupling medium to create cavitation bubbles that expand and contract in the coupling medium.

Referring again to FIG. 2, according to one method, the meter assembly 100 is used for continual, transdermal monitoring of an analyte (e.g., glucose). In a continual monitoring system, the meter assembly 100 measures an analyte concentration (e.g., glucose) at regular intervals, which may range from milliseconds to minutes, to hours. Because the meter 111 may remain coupled to the sensor support 112 for extended periods of time, it is desirable that the meter 111 be of a compact size to minimize the bulkiness and inconvenience to a user. The meter 111 may also be adapted to wirelessly transmit testing data to, for example, a remote computer data management system.

As discussed above, the hydrogel generally includes a monomer and/or monomers and a solvent. In addition to a monomer and solvent, it is contemplated that the hydrogel composition may include other materials. For example, an electrolyte may be added to the hydrogel composition. The electrolyte desirably contains a high salt concentration that assists in exerting osmotic pressure on the skin. By exerting osmotic pressure on the skin, the electrolyte assists in driving ISF to form the liquid diffusion bridge with liquid in the hydrogel. Non-limiting examples of electrolytes that may be used include sodium and potassium salts of chloride, phosphate, citrate, acetate, and lactate.

The hydrogel might also composed of a liquid that contains just enough electrolytes to insure the functionality of the analysis process but hypotonic in comparison to the body fluids such as ISF. That causes a diffusional driving force of numerous solutes into the hypotonic space. That driving force will enhance the transport of glucose toward the sensor surface. The liquid in the hydrogel could also be of a composition that will maximize the efficiency of the reactions that are involved in the analysis process. One example would be a buffer of the optimum pH for the GOx conversion of glucose in the gel.

The hydrogel composition may further include an enzyme to assist in determining the analyte concentration. Depending on the analyte, an enzyme may assist in converting the analyte into a species amenable to detection, such as electrochemical detection. One example of an enzyme that may be used in determining glucose is glucose oxidase. It is contemplated that other enzymes may be used, such as glucose dehydrogenase. If other analytes are of interest, an appropriately selected enzyme may assist in determining the concentration of that analyte.

As discussed above, the test sensor assembly 10 has a dual test sensor 14 containing two independent test sensors 26a, 26b. Having two independent test sensors 26a, 26b allows the test sensor assembly 10 to perform two tests simultaneously. Thus, according to one embodiment, a first sensor 26a performs a first test of an analyte, while a second test sensor 26b performs a second test of the same analyte. In this manner, the second sensor 26b may serve as a failsafe to the first sensor 26a in the event that a malfunction occurs with the first sensor 26a that causes the first sensor 26a to cease functioning, or to give inaccurate results.

According to another embodiment of the present invention, a dual test sensor has a first test sensor and a second test sensor adapted to test for a single analyte, but each of the test sensors contain a different reagent. For example, according to one non-limiting example for glucose monitoring, a first test sensor may contain glucose oxidase as a reagent, while a second test sensor contains glucose dehydrogenase as a reagent. Thus, the use of different reagents within each test sensors allows the second test sensor to serve as a check to verify the results of the first test sensor. An embodiment utilizing different reagents would be particularly useful for long term assays such as continuous glucose monitoring systems (CGMS) where interference effects may build up over time.

According to another embodiment of the present invention, a dual test sensor may be used to identify and monitor a change in flux of an analyte of interest through the user's skin. As previously described, a user's skin may be pre-treated to enhance permeability. Over time, a reduction in the flux, via diffusion, of the analyte of interest through the user's skin may occur. Additionally, the flux at each test sensor location of a dual test sensor may be different. However, a dual test sensor allows the change of flux to be accounted for, by comparing the ratio each sensor over time, as the change in flux will occur at each test sensor location.

According to a further embodiment of the present invention, a dual test sensor may be used to test skin porosity, hydration, and any changes in contact of the dual sensors with the skin during testing by passing a low-level current through the skin from a first sensor to a second sensor. By monitoring skin porosity, hydration, and contact changes, analyte diffusion changes may be corrected for in test results. Additionally, conductivity measured between test sensors may be used to determine that skin porosity has changed, and the sensor assembly needs to be replaced. Monitoring of skin porosity is particularly beneficial when the dual test sensor assembly is used as part of a closed loop artificial pancreas system.

According to yet another embodiment of the present invention, a dual test sensor assembly has a first test sensor adapted to test the analyte of interest, such as, glucose, while a second test sensor is adapted to monitor the functioning of the dual test sensor assembly to detect malfunctions.

According to yet a further embodiment of the present invention, a dual test sensor comprises a first test sensor containing a first reagent adapted to test a first analyte, and a second test sensor containing a second reagent adapted to test a second analyte. Providing a dual test sensor capable of testing for two analytes allows a user to perform two transdermal tests concurrently, easing the testing process for the user. Additionally, results provided from the first sensor may be compared with results provided from the second sensor to correct for variations in flux of the first analyte. Comparing the results from the first and second sensors may be particularly useful when the first analyte level does not effect the second analyte level. According to one non-limiting example, the first analyte may be glucose and the second analyte may be creatinine. If the level of creatinine has not changed, the flux of the creatinine has not changed, thus any glucose level change that occurred is based on a change in glucose level, not a change in flux of glucose.

According to still yet another embodiment of the present invention, a dual test sensor assembly comprises a first test sensor having a first reagent adapted to test a first analyte, and a second test sensor having a second reagent adapted to test a second analyte that may be used to correct interferences that may affect results of the testing of the first analyte. According to one non-limiting example, the first test sensor may contain glucose oxidase as the first reagent, and the second test sensor may contain glucose hydroginase as the second reagent.

According to still yet a further embodiment of the present invention, a dual test sensor assembly comprises a first test sensor that has a first membrane, and a second test sensor that has a second membrane. The first and the second membranes are adapted to remove interfering substances. For example, antibodies may be used that bind the interferences to the membranes. Once the first and second membranes remove the interfering substances, the results of the first and second test sensors may be compared, and the analyte level may be corrected based on the comparison between the two sensors to give the user an accurate test result.

### Alternative Embodiment A

A transdermal test sensor assembly adapted to assist in determining at least one analyte concentration of a fluid sample, the test sensor assembly comprising:

a sensor support;

a first test sensor being coupled to the sensor support;

a second test sensor being coupled to the sensor support;

a first hydrogel composition positioned on the first test sensor; and

a second hydrogel composition positioned on the second test sensor.

### Alternative Embodiment B

The assembly of Alternative Embodiment A, wherein the first test sensor and the second test sensor are adapted to determine a concentration of the same analyte.

### Alternative Embodiment C

The assembly of Alternative Embodiment B, wherein the first test sensor contains a reagent adapted to react with glucose.

### Alternative Embodiment D

The assembly of Alternative Embodiment A, wherein the first test sensor is adapted to determine a concentration of a first analyte and the second test sensor is adapted to determine a concentration of a second analyte.

### Alternative Embodiment E

The assembly of Alternative Embodiment D, wherein the first test sensor contains a first reagent adapted to react with glucose.

### Alternative Embodiment F

The assembly of Alternative Embodiment A, wherein the first test sensor contains a first reagent and the second test sensor contains a second reagent.

### Alternative Embodiment G

The assembly of Alternative Embodiment F, wherein the first reagent is glucose oxidase, and wherein the second reagent is glucose dehydrogenase.

### Alternative Embodiment H

The assembly of Alternative Embodiment F, wherein the first reagent and the second reagent are the same.

### Alternative Embodiment I

The assembly of Alternative Embodiment F, wherein the first reagent and the second reagent are different.

### Alternative Embodiment J

A transdermal analyte-testing assembly adapted to determine a concentration of at least one analyte of a sample, the analyte-testing assembly comprising:

a sensor support;

a first test sensor being coupled to the sensor support;

a second test sensor being coupled to the sensor support;

a first hydrogel composition positioned on the first test sensor;

a second hydrogel composition positioned on the second test sensor; and

an analyte-testing instrument coupled to the sensor support, the analyte-testing instrument being adapted to determine a concentration of at least one analyte of a sample.

### Alternative Embodiment K

The assembly of Alternative Embodiment J, wherein the first test sensor and the second test sensor are adapted to determine the concentration of the same analyte.

### Alternative Process L

A non-invasive method of determining a concentration of at least one analyte in a body fluid, the method comprising the acts of:

providing a dual transdermal test sensor assembly including a sensor support, a first test sensor, a first hydrogel composition, a second test sensor, and a second hydrogel composition, the first test sensor and the second test sensor being coupled to the sensor support;

contacting the transdermal sensor assembly to an area of skin such that the first hydrogel composition and the second hydrogel composition are positioned between the skin and the test sensor;

coupling an analyte-testing instrument to the dual transdermal test sensor assembly; and

determining the concentration of at least one analyte using the analyte-testing instrument.

### Alternative Process M

The method of Alternative Process L, wherein the area of skin is pre-treated.

### Alternative Process N

The method of Alternative Process L, wherein the act of determining the concentration of the at least one analyte using the analyte testing instrument is repeated at pre-selected time intervals.

### Alternative Process O

The method of Alternative Process L, further comprising the acts of:

comparing test results from the first test sensor to test results from the second test sensor; and

calculating a change in flux of at least one analyte using the analyte testing instrument from the act of comparing results.

### Alternative Process P

The method of Alternative Process L, further comprising the acts of:

passing a current from the first test sensor to the second test sensor; and

calculating skin porosity based on the act of passing current.

### Alternative Process Q

The method of Alternative Process L, further comprising the act of determining the concentration of at least a second analyte using the analyte-testing instrument.

### Alternative Process R

The method of Alternative Process L, wherein the second test sensor is adapted to monitor the functioning of dual transdermal test sensor assembly.

### Alternative Process S

The method of Alternative Process L, wherein the first test sensor contains a first reagent adapted to determine a first concentration of a first analyte, and the second test sensor contains a second reagent adapted to determine a second concentration of second analyte.

### Alternative Process T

The method of Alternative Process S, wherein the first reagent and the second reagent are identical.

### Alternative Process U

The method of Alternative Process S, wherein the first reagent and the second reagent are different.

### Alternative Process V

The method of Alternative Process U, wherein the first reagent is glucose oxidase, and the second reagent is glucose dehydrogenase.

### Alternative Process W

The method of Alternative Process S, wherein the first analyte is glucose, and the second analyte is other than glucose.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. A transdermal test sensor assembly adapted to assist in determining at least one analyte concentration of a fluid sample, the test sensor assembly comprising:
a sensor support;
a first test sensor being coupled to the sensor support;
a second test sensor being coupled to the sensor support;
a first hydrogel composition positioned on the first test sensor; and
a second hydrogel composition positioned on the second test sensor
means for inhibiting movement of the first and second test sensors relative to the sensor support.

2. The assembly of claim 1, wherein the first test sensor and the second test sensor are adapted to determine a concentration of the same analyte.

3. The assembly of claim 2, wherein the first test sensor contains a reagent adapted to react with glucose.

4. The assembly of claim 1, wherein the first test sensor is adapted to determine a concentration of a first analyte and the second test sensor is adapted to determine a concentration of a second analyte.

5. The assembly of claim 4, wherein the first test sensor contains a first reagent adapted to react with glucose.

6. The assembly of claim 1, wherein the first test sensor contains a first reagent and the second test sensor contains a second reagent.

7. The assembly of claim 6, wherein the first reagent is glucose oxidase, and wherein the second reagent is glucose dehydrogenase.

8. The assembly of claim 6, wherein the first reagent and the second reagent are the same.

9. The assembly of claim 6, wherein the first reagent and the second reagent are different.

10. A transdermal analyte-testing assembly adapted to determine a concentration of at least one analyte of a sample, the analyte-testing assembly comprising:
a sensor support;
a first test sensor being coupled to the sensor support;
a second test sensor being coupled to the sensor support;
a first hydrogel composition positioned on the first test sensor;
a second hydrogel composition positioned on the second test sensor;
an analyte-testing instrument coupled to the sensor support, the analyte-testing instrument being adapted to determine a concentration of at least one analyte of a sample, and
means for inhibiting movement of the first and second test sensors relative to the sensor support.

11. The assembly of claim 10, wherein the means for inhibiting movement of the first and second test sensors relative to the sensor support include a recessed area having dimensions generally similar to the dimensions of the first and second test sensors, a flexible element adapted to attach to the sensor support, an adhesive positioned between the first and second test sensors and the sensor support.

12. A non-invasive method of determining a concentration of at least one analyte in a body fluid, the method comprising the acts of:
pretreating and disrupting an area of the skin with ultrasound energy to increase the skin permeability;
providing a dual transdermal test sensor assembly including a sensor support, a first test sensor, a first hydrogel composition, a second test sensor, and a second hydrogel composition, the first test sensor and the second test sensor being coupled to the sensor support;
contacting the transdermal sensor assembly to the area of skin such that the first hydrogel composition and the second hydrogel composition are positioned between the skin and the test sensor;
coupling an analyte-testing instrument to the dual transdermal test sensor assembly; and
determining the concentration of at least one analyte using the analyte-testing instrument.

13. (canceled)

14. The method of claim 12, wherein the act of determining the concentration of the at least one analyte using the analyte-testing instrument is repeated at pre-selected time intervals.

15. The method of claim 12, further comprising the acts of:
comparing test results from the first test sensor to test results from the second test sensor; and
calculating a change in flux of at least one analyte using the analyte testing instrument from the act of comparing results.

16. The method of claim 12, further comprising the acts of:
passing a current from the first test sensor to the second test sensor; and
calculating skin porosity based on the act of passing current.

17. The method of claim 12, further comprising the act of determining the concentration of at least a second analyte using the analyte-testing instrument.

18. (canceled)

19. The method of claim 12, wherein the first test sensor contains a first reagent adapted to determine a first concentration of a first analyte, and the second test sensor contains a second reagent adapted to determine a second concentration of second analyte.

20. The method of claim 19, wherein the first reagent and the second reagent are identical.

21. The method of claim 19, wherein the first reagent and the second reagent are different.

22. The method of claim 21, wherein the first reagent is glucose oxidase, and the second reagent is glucose dehydrogenase.

23. The method of claim 19, wherein the first analyte is glucose, and the second analyte is other than glucose.

24. The assembly of claim 1 wherein the means for inhibiting movement of the first and second test sensors relative to the sensor support include a recessed area having dimensions generally similar to the dimensions of the first and second test sensors, a flexible element adapted to attach to the sensor support, an adhesive positioned between the first and second test sensors and the sensor support.

25. The assembly of claim 24 wherein the means for inhibiting movement of the first and second test sensors relative to the sensor support include the recessed area having dimensions generally similar to the dimensions of the first and second test sensors, the recessed area including extensions therefrom, the first and second test sensors forming respective apertures, the extensions extending through respective apertures to assist in maintaining the location of the first and second test sensors.
